(19) United States
(12) Patent Application Publication
(10) Pub. No.: US 2024/0216364 A1
(43) Pub. Date: Jul. 4, 2024

(54) STRONG POTENTIATION OF TLR3 AGONISTS EFFECTS USING FXR AGONISTS AS A COMBINED TREATMENT

(71) Applicant: ENYO PHARMA, LYON (FR)

(72) Inventors: RAPHAËL DARTEIL, LYON (FR); DAVID DURANTEL, GENAS (FR)

(21) Appl. No.: 18/288,393

(22) PCT Filed: Apr. 28, 2022
(86) PCT No.: PCT/EP2022/061296

§ 371 Date: Oct. 26, 2023

(30) Foreign Application Priority Data
Apr. 28, 2021 (EP) 21170970.4

(51) Int. Cl. A61K 31/496; A61K 31/222; A61K 31/4162; A61K 31/421; A61K 31/4439; A61K 31/46; A61K 31/575; A61K 31/713; A61P 31/20

(52) U.S. Cl. CPC A61K 31/496; A61K 31/222; A61K 31/4162; A61K 31/421; A61K 31/4439; A61K 31/46; A61K 31/575; A61K 31/713; A61P 31/20

(57) ABSTRACT

The present invention relates to a combination of a FXR agonist and a TLR3 agonist having a synergistic effect and its use for the treatment of disease and disorders.

### FIELD OF THE INVENTION

The present invention relates to the field of medicine, in particular the treatment of diseases and disorders such as an infection, especially a viral infection, a bacterial infection or a protozoan infection, a cancer, an autoimmune disease and an inflammatory disease.

### BACKGROUND OF THE INVENTION

Toll-like receptor 3 (TLR3) is a pattern recognition receptor that senses exogenous (viral) as well as endogenous (mammalian) double-stranded RNA in endosomes. Upon agonization with cognate ligands or agonists, TLR3 dimerizes and initiates a signal transduction pathway that culminates with the secretion of pro-inflammatory cytokines, including type-I interferon (IFN). The latter is essential not only for innate immune responses to infection but also for the initiation of antigen-specific immunity against viruses and malignant cells. These aspects of TLR3 biology have supported the development of various agonists for use as stand-alone agents or combined with other therapeutic modalities.

Polyl:C and polyA:U were originally synthesized in the mid-1960s. Several modified versions of polylC were developed in the 1970s'. Then a first modified polylC dsRNA (Ampligen™ or rintatolimod) by substituting a uridylic acid at a molar ratio of 12:1 in the synthesis of the polycytidylic acid strand resulting in a double-stranded molecule with occasional mismatches and a much more rapid metabolism in vivo (polyl:polyC12U; polylC12U) has been described (Carter et al, 1972, J. Mol. Biol.70(3),567-587). Another modified polylC by stabilizing the molecule with polylysine and formulating it with carboxymethylcellulose (polylCLC, best known as Hiltonol™) has also been developed (Levy et al, J. Infect. Dis.132(4),434-439 (1975)). In addition to these two historical modified polylC compounds, among others, Riboxxol (also known as RGIC®50) is a synthetic dsRNA containing cytosines, inosines and guanosines (Naumann et al, Clin Dev Immunol., 2013, 2013, 283649) and ARNAX is a TLR3 agonist originally developed by Matsumoto and collaborators that consists of a phosphorothioate oligodeoxynucleotide (ODN)-guided dsRNA (Matsumoto et al, Nat Commun. 2015, 6, 6280). Two additional TLR3 agonists, IPH3102 and TL-532, are respectively in preclinical development at Innate Pharma (Marseille, France) and Tollys (Lyon, France).

TLR3 agonists are suggested to be used as vaccine adjuvants (e.g., WO20191361, WO20030634, WO17083963, WO15035128), for instance in vaccine against cancer (e.g., WO19195626, WO18085734) or virus such as HBV (e.g., WO21067181), HIV (e.g., WO21011544, WO20236753), RSV (e.g., WO18109220), influenza (e.g., WO14085580), papillomavirus (e.g., WO12006727) or parasites such as Eimeria (e.g., WO18115229).

For the treatment of cancer, combinations of a TLR3 agonist with a therapeutic antibody targeting for instance OX40, 4-1BB, PD-1, PD-L1, TIM3, CTLA-4 or CD73 (e.g., WO20128893, WO20077077, WO19173692, WO17079431, WO17024296, WO16019472, WO15168379) or Adoptive Cell Therapy (e.g., WO20072366) have been disclosed. They have also been described for their use for the treatment of cancer by inducing apoptosis (e.g., WO18087323, WO10012965).

More specifical, clinical trials are ongoing with Ampligen™ for colorectal cancer, melanoma and prostate cancer combined with anti-PD-1 inhibitors such as pembrolizumab, anti-PD-L1 inhibitors, COX2 inhibitors such as celecoxib or aspirin and/or IFNα-2b; with Hiltonol for breast cancer, melanoma, mesothelioma, prostate cancer alone or combined with anti-PD-1 inhibitors such as pembrolizumab or nivolumab, radiotherapy, FLT3LG (fms-like tyrosine kinase 3 ligand), multipeptide vaccine, and/or anti-CD27 agonist such as varlilumab.

TLR3 agonists have also been suggested for the treatment of infection, in particular viral infection, alone or in combination with an antiviral agent, an antibody and the like (e.g., WO20010107 or WO19226829, EP0213921 for HIV treatment).

TLR3 agonists could be used for the treatment of degenerative inflammatory process (e.g., WO07089151), for the treatment of multiple sclerosis in combination with natalizumab (e.g., WO19169317) and they have further been developed for the treatment of chronic fatigue syndrome and impaired physical performance (e.g., WO10042229).

More particularly, the effects of TLR3 agonist for the treatment of HBV infection have been reported (Lucifora J et al. Sci Rep. 2018 Mar. 29; 8(1):5390). In this article, the authors demonstrated that agonist of TLR3 such as poly(I:C)-(HMW) or Riboxxol activated hepatocytes (PHH or dHepaRG) innate responses and efficiently decreased levels of all HBV replication markers, including a strong phenotype on HBV RNAs. See also, Ma et al, Vaccines, 2018, 6, 6.

Then, TLR3 agonists are used in several therapeutic indications but their uses can still be improved.

### SUMMARY OF THE INVENTION

The inventors have observed the surprising potentiating effect of an FXR (farnesoid X receptor) agonist on the activity of a TLR3 agonist, especially on the HBV replication markers. Indeed, at least a strong potentiating effect, and even a synergistic effect, is reported for the combination of a TLR3 agonist and an FXR agonist. Accordingly, a FXR agonist can be used in combination with a TLR3 agonist to increase the effect of the TLR3 agonist. Alternatively, a TLR3 agonist can be used in combination with an FXR agonist to increase the effect of the FXR agonist. Thus, the combination of a TLR3 agonist with an FXR agonist can be used for the treatment of any disease or disorder susceptible to be treated with a TLR3 or FXR agonist. Without being bound by a theory, it is hypothesized that the combination has a strong potentiating effect or synergistic effect to activate innate immunity, in particular interferon production. The synergistic effect has been observed with several FXR agonists having different structures and with several unrelated TLR3 agonists. Thereby, the synergistic effect is supported by the activity of FXR agonists and TLR3 agonists and is not specific of a particular structure.

Accordingly, the present invention relates to a pharmaceutical composition comprising an FXR agonist and a TLR3 agonist and its use for the treatment of a disease, to a pharmaceutical composition comprising an FXR agonist for use in combination with a TLR3 agonist for the treatment of a disease, and pharmaceutical composition comprising a TLR3 agonist for use in combination with an FXR agonist for the treatment of a disease. In particular, the FXR agonist and the TLR3 agonist are used so as to obtain a synergistic effect for activate innate immunity, in particular interferon production.

In one aspect, the FXR agonist is selected from the group consisting of EYP001 (Vonafexor), (UN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502. In a very specific aspect, the FXR agonist is EYP001 (Vonafexor). In a very specific aspect, the FXR agonist is selected from the group consisting of EYP001 (Vonafexor), UN452 (Tropifexor), LMB763 (Nidufexor), GS-9674 (Cilofexor), GW4064, Fexaramine and OCA (Ocaliva).

In another aspect, the TLR3 agonist is a double stranded RNA compound (dsRNA) or a derivative thereof. The TLR3 agonist can be selected in the group consisting of Poly I:C (polyribosinic:polyribocytidic acid), polyA:U (poly(adenylic acid-uridylic acid), Poly ICLC (polyinosinic acid-polycytidylic acid-poly-L-lysinecarboxy-methylcellulose complex or Hiltonol), Polyl:polyC₁₂U (polylC₁₂U, Ampligen or Rintatolimod), Riboxxol (RGIC®50), RIBOXXIM (RGIC®100), APOXXIM, ARNAX, IPH3102, MCT-465 and MCT-485. In a very specific aspect, TLR3 agonist is Rintatolimod, Hiltonol or Riboxxol. In a very specific aspect, the TLR3 agonist is a Poly I:C (polyribosinic:polyribocytidic acid). In another very specific aspect, TLR3 agonist is Riboxxol.

In a very specific aspect, the TLR3 agonist selected from the group consisting of Riboxxol, Rintatolimod, and Hiltonol and the FXR agonist is selected from the group consisting of EYP001 (Vonafexor), UN452 (Tropifexor), LMB763 (Nidufexor), GS-9674 (Cilofexor), GW4064, Fexaramine and OCA (Ocaliva).

In another very specific aspect, the TLR3 agonist is Riboxxol and the FXR agonist is selected from the group consisting of EYP001 (Vonafexor), UN452 (Tropifexor), LMB763 (Nidufexor), GS-9674 (Cilofexor), GW4064, Fexaramine and OCA (Ocaliva).

In another very specific aspect, the TLR3 agonist is Rintatolimod, Hiltonol or Riboxxol and the FXR agonist is EYP001 (Vonafexor). More specifically, the TLR3 agonist is Riboxxol and the FXR agonist is EYP001 (Vonafexor).

Optionally, the disease to be treated is selected from the group consisting to an infection, especially a viral infection, a bacterial infection or a protozoan infection, a cancer, and an autoimmune disease. In particular, the FXR agonist and the TLR3 agonist are used so as to obtain a synergistic effect for the treatment of a disease selected from the group consisting to an infection, especially a viral infection, a bacterial infection or a protozoan infection, a cancer, and an autoimmune disease.

Optionally, the disease is an infection by a virus selected from the group consisting of hepatotropic virus including hepatitis A virus, hepatitis B virus, hepatitis C virus, hepatitis D virus, hepatitis E virus, Herpesviridae virus including herpes simplex virus (HSV), varicella-zoster virus, Kaposis sarcoma herpesvirus and cytomegalovirus (CMV), Hepadnaviridae virus including HBV, papillomavirus (HPV), coronavirus including SARS-Cov1, MERS-Cov and SARS-Cov2, retrovirus including HIV, influenza virus and rhinoviruses. In a preferred aspect, the virus is HBV, HDV, and SARS-Cov2. In a specific aspect, the disease is a hepatitis B virus infection and/or a hepatitis D virus infection, especially a chronic HBV infection and/or a chronic HDV infection. In a very specific aspect, the disease is a hepatitis B virus infection, especially a chronic HBV infection.

Optionally, the disease is Chronic fatigue syndrome.

Optionally, the disease is a cancer, in particular a solid cancer or a hematopoietic cancer, especially selected from the group consisting of AIDS-related Kaposi's sarcoma, leukemia such as hairy-cell leukemia, chronic myeloid leukemia, and non-Hodgkin's leukemia, lymphoma such as follicular lymphoma, B-cell lymphoma, cutaneous T-cell lymphoma and adult T-cell leukemia-lymphoma, carcinoid tumors, melanoma, multiple myeloma, renal cell carcinoma, colorectal adenocarcinoma, hepatocarcinoma, breast cancer, prostate cancer, ovarian cancer, pancreas cancer, peritoneal cancer, bladder cancer, lung cancer, glioblastoma, oral carcinoma, glioma, head and neck cancer, sarcoma, and neuroendocrine tumors.

Optionally, the disease is an autoimmune disease, especially an autoimmune disease selected from the group consisting of multiple sclerosis, rheumatoid arthritis, Behçet's syndrome, Churg-Strauss syndrome, Guillain-Barre syndrome, and inflammatory bowel disease including ulcerative colitis and Crohn's disease.

### BRIEF DESCRIPTION OF THE FIGURES

FIG. 1. Synergistic effect of the combination of an FXR agonist and a TLR3 agonist on HBV replication in infected fresh primary human hepatocytes (PHH) assessed by HBsAg.

FIG. 2. Synergistic effect of the combination of an FXR agonist and a TLR3 agonist on HBV replication in infected fresh primary human hepatocytes (PHH) assessed by HBeAg.

FIG. 3. Synergistic effect of the combination of an FXR agonist and a TLR3 agonist on HBV replication in infected fresh primary human hepatocytes (PHH) assessed by secreted HBV DNA.

FIG. 4. Synergistic effect of the combination of an FXR agonist with two different TLR3 agonists on HBV replication in infected fresh primary human hepatocytes (PHH) assessed by HBsAg and HBeAg.

FIG. 5. Synergistic effect of the combination of six different FXR agonists with a TLR3 agonist on HBV replication in infected fresh primary human hepatocytes (PHH) assessed by HBsAg and HBeAg.

### DETAILED DESCRIPTION OF THE INVENTION

The present invention relates to a pharmaceutical composition comprising an FXR agonist and a TLR3 agonist, and their use for the treatment of a disease. In particular, the FXR agonist and the TLR3 agonist are used so as to obtain a synergistic effect to activate innate immunity, in particular interferon production.

In addition, the present invention relates to the use of an FXR agonist to potentiate the effect of a TLR3 agonist, in particular on the activation of innate immunity, in particular interferon production. More specifically, it relates to the use of an FXR agonist to potentiate the effect of a TLR3 agonist on a viral infection, especially on an infection by HBV. Accordingly, the present invention relates to a pharmaceutical composition comprising an FXR agonist for use in combination with a TLR3 agonist for the treatment of a disease, the use of a pharmaceutical composition comprising an FXR agonist for the manufacture of a medicine to be used in combination with a TLR3 agonist for the treatment of a disease. It relates to a method for treating a disease in a subject, comprising administering a therapeutic effective amount of a pharmaceutical composition comprising a TLR3 agonist and administering a therapeutic effective amount of a pharmaceutical composition comprising an FXR agonist. Optionally, said TLR3 agonist and FXR agonist can be in the same pharmaceutical composition and the method may comprises administering a therapeutic effective amount of a pharmaceutical composition comprising a TLR3 agonist and an FXR agonist.

Finally, the present invention relates to the use of a TLR3 agonist to potentiate the effect of an FXR agonist, in particular on the activation of innate immunity, in particular interferon production. Accordingly, the present invention relates to a pharmaceutical composition comprising a TLR3 agonist for use in combination with an FXR agonist for the treatment of a disease.

Accordingly, the present invention relates to
a pharmaceutical composition comprising an FXR agonist and a TLR3 agonist, and optionally a pharmaceutically acceptable carrier and/or an additional active ingredient, in particular for use in the treatment of a disease, preferably with the FXR agonist and TLR3 agonist being used so as to obtain a potentiating or synergistic effect for activating the innate immunity, in particular interferon production, and especially for decreasing the HBV replication; optionally, the pharmaceutical composition may comprise at least one additional active ingredient;
a product or kit containing an FXR agonist or a pharmaceutical composition comprising it and a TLR3 agonist as a combined preparation for simultaneous, separate or sequential use, in particular in the treatment of a disease, preferably with the FXR agonist and TLR3 agonist being used so as to obtain a potentiating or synergistic effect for activating the innate immunity, in particular interferon production, and especially for decreasing the HBV replication; optionally, the product or kit may comprise at least one additional active ingredient;
a combined preparation which comprises an FXR agonist or a pharmaceutical composition comprising it and a TLR3 agonist for simultaneous, separate or sequential use, in particular in the treatment of a disease, preferably with the FXR agonist and TLR3 agonist being used so as to obtain a potentiating or synergistic effect for activating the innate immunity, in particular interferon production, and especially for decreasing the HBV replication; optionally, the combined preparation may comprise at least one additional active ingredient;
a pharmaceutical composition comprising an FXR agonist for the use in the treatment of a disease in combination with a treatment with a TLR3 agonist, preferably with the FXR agonist and the TLR3 agonist being used so as to obtain a potentiating or synergistic effect for activating the innate immunity, in particular interferon production, and especially for decreasing the HBV replication; optionally, the pharmaceutical composition may comprise at least one additional active ingredient;
a pharmaceutical composition comprising a TLR3 agonist for the use in the treatment of a disease, in combination with a treatment with an FXR agonist, preferably with the FXR agonist and the TLR3 agonist being used so as to obtain a potentiating or synergistic effect for activating the innate immunity, in particular interferon production, and especially for decreasing the HBV replication; optionally, the pharmaceutical composition may comprise at least one additional active ingredient;
the use of a pharmaceutical composition comprising an FXR agonist for the manufacture of a medicament for the treatment of a disease in combination with a treatment with TLR3 agonist, preferably with the FXR agonist and the TLR3 agonist being used so as to obtain a potentiating or synergistic effect for activating the innate immunity, in particular interferon production, and especially for decreasing the HBV replication; optionally, the pharmaceutical composition may comprise at least one additional active ingredient;
the use of a pharmaceutical composition comprising a TLR3 agonist for the manufacture of a medicament for the treatment of a disease in combination with a treatment with an FXR agonist, preferably with the FXR agonist and the TLR3 agonist being used so as to obtain a potentiating or synergistic effect for activating the innate immunity, in particular interferon production, and especially for decreasing the HBV replication; optionally, the pharmaceutical composition may comprise at least one additional active ingredient;
the use of a pharmaceutical composition comprising an FXR agonist and a TLR3 agonist, and optionally a pharmaceutically acceptable carrier for the manufacture of a medicament for the treatment of a disease, especially chronic hepatitis B, preferably with the FXR agonist and TLR3 agonist are used so as to obtain a potentiating or synergistic effect for activating the innate immunity, in particular interferon production, and especially for decreasing the HBV replication; optionally, the pharmaceutical composition may comprise at least one additional active ingredient;
a method for treating a disease in a subject in need thereof, comprising administering an effective amount of a pharmaceutical composition comprising a) an FXR agonist, b) a TLR3 agonist, and a pharmaceutically acceptable carrier, preferably with the FXR agonist and the TLR3 agonist being used so as to obtain a potentiating or synergistic effect for activating the innate immunity, in particular interferon production, and especially for decreasing the HBV replication; optionally, the pharmaceutical composition may comprise at least one additional active ingredient or the method may further comprise the administration of at least one additional active ingredient;
a method for treating a disease in a subject in need thereof, comprising administering an effective amount of a pharmaceutical composition comprising an FXR agonist, and an effective amount of a pharmaceutical composition comprising a TLR3 agonist, preferably with the FXR agonist and the TLR3 agonist being used so as to obtain a potentiating or synergistic effect for activating the innate immunity, in particular interferon production, and especially for decreasing the HBV replication; optionally, one of the pharmaceutical compositions may comprise at least one additional active ingredient or the method may further comprise the administration of at least one additional active ingredient.

The TLR3 agonist and the FXR agonist can be selected among any and all specific TLR3 agonists and FXR agonists disclosed herein. The disease can be any disease and disorder disclosed herein. The TLR3 agonist can be used at a therapeutic or sub-therapeutic amount. The FXR agonist can be used at a therapeutic or sub-therapeutic amount.

### Definition

The term “FXR” refers to the farnesoid X receptor, which is a nuclear receptor that is activated by supraphysiological levels of farnesol (Forman et al., Cell, 1995,81,687-693). FXR, is also known as NR1H4, retinoid X receptor-interacting protein 14 (RIP14) and bile acid receptor (BAR). Containing a conserved DNA-binding domain (DBD) and a C-terminal ligand-binding domain (LBD), FXR binds to and becomes activated by a variety of naturally occurring bile acids (BAs), including the primary bile acid chenodeoxycholic acid (CDCA) and its taurine and glycine conjugates. Upon activation, the FXR-RXR heterodimer binds the promoter region of target genes and regulates the expression of several genes involved in bile acid homeostasis. Hepatic FXR target genes fall into two main groups. The first group functions to decrease hepatic bile acids concentrations by increasing export and decreasing their synthesis. The second group of FXR target genes such as the phospholipid transport protein PLTP and apolipoproteins modulates lipoprotein levels in the serum and decreases plasma triglyceride concentration. For a more detailed list of FXR-regulated genes, see, e.g., WO 03/016288, pages 22-23. U.S. Pat. No. 6,005,086 discloses the nucleic acid sequence coding for a mammalian FXR protein. Human FXR is described in Uniprot under accession number Q96RI1. The human polypeptide sequences for FXR are deposited in nucleotide and protein databases under accession numbers NM_005123, Q96RI1, NP_005114 AAM53551, AAM53550, AAK60271.

In this specification, the term “FXR agonist” has its general meaning in the art and refers in particular to compounds that function by targeting and binding the farnesoid X receptor (FXR) and which activate FXR by at least 40% above background in the assay described in Maloney et al. (J. Med. Chem. 2000, 43:2971-2974).

In some embodiments, the FXR agonist of the invention is a selective FXR agonist. As used herein, the term “selective FXR agonist” refers to an FXR agonist that exhibits no significant cross-reactivity to one or more, ideally substantially all, of a panel of nuclear receptors consisting of LXRα, LXRβ, PPARα, PPARγ, PPARδ, RXRα, RARγ, VDR, PXR, ERα, ERβ, GR, AR, MR and PR. Methods of determining significant cross-reactivity are described in J. Med. Chem. 2009, 52, 904-907.

The term “TLR3” refers to Toll-like receptor 3 (TLR3) also known as CD283. Human TLR3 is described in Uniprot under accession number 015455. The human polypeptide sequences for TLR3 are deposited in nucleotide and protein databases under accession numbers NM_003265.2 and NP_003256.1, respectively.

The term “TLR3 agonist” refers to an affinity agent (i.e., a molecule that binds a target molecule) capable of activating a TLR3 polypeptide to induce a full or partial receptor-mediated response. For example, an agonist of TLR3 induces TLR3-mediated signaling, either directly or indirectly. A TLR3 agonist, as used herein, may or may not interact directly with the TLR3 polypeptide. A “nucleotide agonist” or “nucleic acid agonist” refers to the situation where the affinity agent comprises or consists of nucleotides and/or nucleic acid(s). An “antibody agonist” refers to the situation where the affinity agent is an antibody. A TLR3 agonist can also be a small molecule. The activation of TLR3 can be measured by several well-known methods, by the person skilled in the art. For instance, the activation can be measured in reporter HEK cells ectopically expressing TLR3 (c.f. https://www.invivogen.com/hek-blue-htlr3).

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease. More particularly, the term “treating”, or “treatment”, means alleviating HBV infection, arresting disease development, and/or removing HBV by administering the composition.

More particularly, the treatment of hepatitis B infection, especially chronic hepatitis B, is shown by a decrease of HBV replication. The HBV replication can be assessed by determining at least one of HBeAg level, HBsAg level, HBcrAg level, pre-genomic RNA (HBV pgRNA) level, pre-core RNA level, relaxed circular DNA (HBV rcDNA) level, HBV cccDNA level or HBV DNA level in the subject. HBsAg loss and seroconversion are generally the goal for clinical functional cure. By decreasing, it is meant that the level in at least one of HBeAg level, HBsAg level, HBcrAg level, pre-genomic RNA (HBV pgRNA) level, pre-core RNA level, relaxed circular DNA (HBV rcDNA) level, HBV cccDNA level and HBV DNA level is decreased in comparison with the absence of treatment.

By decreasing HBV replication, it is preferably meant that the HBV replication is decreased by at least 10 or 100 fold in comparison with the HBV replication in absence of treatment. For instance, the HBV replication can be assessed by determining the HBV DNA level and this level is decreased by at least 10 or 100 fold in comparison with the HBV replication in absence of EYP001. Alternatively, HBV cccDNA level is decreased by at least 10, 15, 20, 25, 30, 35, 40, 45 or 50% in comparison with the absence of treatment.

As used herein, the terms “subject”, “individual” or “patient” are interchangeable and refer to a human, including adult, child, newborn and human at the prenatal stage. In a particular aspect, the subject or patient suffers of hepatitis B infection, in particular a chronic hepatitis B.

The terms “quantity,” “amount,” and “dose” are used interchangeably herein and may refer to an absolute quantification of a molecule.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “therapeutically effective amount” refers to a quantity of an active ingredient or of a pharmaceutical composition, which prevents, removes or reduces the deleterious effects of the disease, particularly infectious disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “sub-therapeutic amount” or “sub-therapeutic dose” refers to a dosage, which is less than that dosage which would produce a therapeutic result in the subject if administered in the absence of the other agent. For instance, “sub-therapeutic amount” or “sub-therapeutic dose” can refer to a dosage which is decreased by 25, 50, 70, 80 or 90% in comparison to the therapeutically effective amount, especially the conventional therapeutic dosage for the same indication and the same administration route when used alone. The conventional therapeutic dosages are those acknowledged by the drug approvals agencies (e.g., FDA or EMEA).

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

The terms “kit”, “product” or “combined preparation”, as used herein, defines especially a “kit of parts” in the sense that the combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

By “a synergistic effect” is intended to refer to an effect, which is more than the sum of the effects of each molecule alone.

In the context of HBV, “a synergistic effect” is intended to refer to an effect for decreasing the HBV replication, which is more than the sum of the effects of each molecule alone. HBV replication can be assessed by determining surface HBV antigen (HBsAg), HBeAg, HBV core related antigen (HBcrAg), HBV DNA, HBV pre-genomic RNA, HBV pre-core RNA and/or HBV cccDNA levels. More particularly, the effect is observed on the pre-genomic RNA (HBV pgRNA) and/or on the hepatitis B core related antigen (HBcrAg).

### FXR Agonist

FXR agonists are well known to the skilled person.

For example, the skilled person may easily identify FXR agonist from the following publications (the disclosure of which being incorporated herein by reference):
Abenavoli L, et al. Pharmaceuticals (Basel). 2018 Oct. 11; 11(4). pii: E104. doi: 10.3390/ph11040104. Review.
Adorini L, et al. Drug Discov Today. 2012 September;17(17-18):988-97. doi: 10.1016/j.drudis.2012.05.012. Epub 2012 May 29. Review.
Akwabi-Ameyaw A, et al. Bioorg Med Chem Lett. 2009 Aug. 15; 19(16):4733-9. doi: 10.1016/j.bmcl.2009.06.062. Epub 2009 Jun. 21.
Akwabi-Ameyaw A, et al. Bioorg Med Chem Lett. 2008 Aug. 1; 18(15):4339-43. doi: 10.1016/j.bmcl.2008.06.073. Epub 2008 Jun. 28.
Akwabi-Ameyaw A, et al. Bioorg Med Chem Lett. 2011 Oct. 15; 21(20):6154-60. doi: 10.1016/j.bmcl.2011.08.034. Epub 2011 Aug. 11.
Baghdasaryan A, et al. Hepatology. 2011 October;54(4):1303-12. doi: 10.1002/hep.24537.
Bass J Y, et al. Bioorg Med Chem Lett. 2009 Jun. 1; 19(11):2969-73. doi: 10.1016/j.bmcl.2009.04.047. Epub 2009 Apr. 18.
Bass J Y, et al. Bioorg Med Chem Lett. 2011 Feb. 15; 21(4):1206-13. doi: 10.1016/j.bmcl.2010.12.089. Epub 2010 Dec. 23.
Buijsman et al., Curr. Med. Chem. 2005, 12, 1017
Carino et al, Sci Rep. 2017 Feb. 16; 7:42801. doi: 10.1038/srep42801.
Chiang P C, et al. J Pharm Sci. 2011 November; 100(11):4722-33. doi: 10.1002/jps.22664. Epub 2011 Jun. 9.
Crawley, Expert Opin. Ther. Pat. 2010, 20, 1047
Feng S, et al. Bioorg Med Chem Lett. 2009 May 1;19(9):2595-8. doi: 10.1016/j.bmcl.2009.03.008. Epub 2009 Mar. 9.
Festa et al, Front Pharmacol. 2017 Mar. 30; 8:162. doi: 10.3389/fphar.2017.00162. eCollection 2017.
Finamore et al, Sci Rep. 2016 Jul. 6; 6:29320. doi: 10.1038/srep29320.
Flatt B, et al. J Med Chem. 2009 Feb. 26; 52(4):904-7. doi: 10.1021/jm8014124.
Gege et al, Curr Top Med Chem. 2014; 14(19):2143-58.
Gege et al, Handbook of Experimental Pharmacology, doi: 10.1007/164_2019_232.
Genin et al, J Med Chem. 2015 Dec. 24; 58(24):9768-72. doi: 10.1021/acs.jmedchem.5b01161. Epub 2015 Dec. 2.
Ghebremariam Y T, et al. PLoS One. 2013 Apr. 4; 8(4):e60653. doi: 10.1371/journal.pone.0060653. Print 2013.
Gioiello A, et al. Bioorg Med Chem. 2011 Apr. 15; 19(8):2650-8. doi: 10.1016/j.bmc.2011.03.004. Epub 2011 Mar. 10.
Hoekstra M, et al. Mol Cell Endocrinol. 2012 Oct. 15; 362(1-2):69-75. doi: 10.1016/j.mce.2012.05.010. Epub 2012 May 27.
Iguchi Y, et al. Steroids. 2010 January;75(1):95-100. doi: 10.1016/j.steroids.2009.11.002. Epub 2009 Nov. 12.
Kinzel et al, Bioorg Med Chem Lett. 2016 Aug. 1; 26(15):3746-53. doi: 10.1016/j.bmcl.2016.05.070. Epub 2016 May 24.
Lin H R. Bioorg Med Chem Lett. 2012 Jul. 15; 22(14):4787-92. doi: 10.1016/j.bmcl.2012.05.057. Epub 2012 May 23.
Lundquist J T, et al. J Med Chem. 2010 Feb. 25; 53(4):1774-87. doi: 10.1021/jm901650u.
Ma Y, et al. Pharm Res. 2013 May; 30(5):1447-57. doi: 10.1007/s11095-013-0986-7. Epub 2013 Feb. 1.
Marinozzi M, et al. Bioorg Med Chem. 2013 Jul. 1; 21(13):3780-9. doi: 10.1016/j.bmc.2013.04.038. Epub 2013 Apr. 23.
Massafra et al. Pharmacol Ther. 2018 November;191:162-177. doi: 10.1016/j.pharmthera.2018.06.009. Epub 2018 Jun. 20.
Misawa T, et al. Bioorg Med Chem Lett. 2012 Jun. 15; 22(12):3962-6. doi: 10.1016/j.bmcl.2012.04.099. Epub 2012 Apr. 30.
Pellicciari et al, J Med Chem. 2016 Oct. 4.
Richter H G, et al. Bioorg Med Chem Lett. 2011 Feb. 15; 21(4):1134-40. doi: 10.1016/j.bmcl.2010.12.123. Epub2010 Dec. 31.
Rizzo G, et al. Mol Pharmacol. 2010 October;78(4):617-30. doi: 10.1124/mol.110.064501. Epub 2010 Jul. 14.
Roda et al, J Pharmacol Exp Ther. 2014 July;350(1):56-68. doi: 10.1124/jpet.114.214650. Epub 2014 May 1.
Schuster D, et al. Bioorg Med Chem. 2011 Dec. 1; 19(23):7168-80. doi: 10.1016/j.bmc.2011.09.056. Epub 2011 Oct. 4.
Schwabl et al, J Hepatol. 2017 April; 66(4):724-733. doi: 10.1016/j.jhep.2016.12.005. Epub 2016 Dec. 18.
Samlley et al, Bioorg Med Chem Lett. 2015 Jan. 15; 25(2):280-4. doi: 10.1016/j.bmcl.2014.11.050. Epub 2014 Nov. 26.
Sepe et al. Expert Opin Ther Pat. 2018 May; 28(5):351-364. doi: 10.1080/13543776.2018.1459569. Epub 2018 Apr. 13. Review.
Sepe et al. Expert Opin Ther Pat. 2015; 25(8):885-96. doi: 10.1517/13543776.2015.1045413. Review.
Soisson S M, et al. Proc Natl Acad Sci USA. 2008 Apr. 8; 105(14):5337-42. doi: 10.1073/pnas.0710981105. Epub 2008 Apr. 7.
Townsend S A, Newsome P N. Aliment Pharmacol Ther. 2017 September;46(5):494-507. doi: 10.1111/apt.14210. Epub2017 Jul. 4.
Tully et al, J Med Chem. 2017 Dec. 28; 60(24):9960-9973. doi: 10.1021/acs.jmedchem.7b00907. Epub 2017 Dec. 8.
Wang et al, J Am Soc Nephrol. 2018 January;29(1):118-137. doi: 10.1681/ASN.2017020222. Epub 2017 Oct. 31.
Wang et al, Bioorg Med Chem Lett. 2017 Aug. 1; 27(15):3386-3390. doi: 10.1016/j.bmcl.2017.06.003. Epub 2017 Jun. 3.
Wang H, et al. Expert Opin Ther Pat. 2018 November; 28(11):765-782. doi: 10.1080/13543776.2018.1527906. Epub 2018 Oct. 8. Review
Watanabe M, et al. J Biol Chem. 2011 Jul. 29;286(30):26913-20. doi: 10.1074/jbc.M111.248203. Epub 2011 Jun. 1.
Yu D, et al. Steroids. 2012 November; 77(13):1335-8. doi: 10.1016/j.steroids.2012.09.002. Epub 2012 Sep. 21.
Zhang S, et al. J Hepatol. 2009 August; 51(2):380-8. doi: 10.1016/j.jhep.2009.03.025. Epub 2009 May 18.

Typically, FXR agonists include the class of steroidal FXR agonists and non-steroidal FXR agonists.

In certain embodiments of the invention, the FXR agonist is selected from small molecule compounds which act as FXR modulators that have been disclosed in the following publications: EP1392714; EP1568706; JP2005281155; US20030203939; US2005080064; US2006128764; US20070015796; US20080038435; US20100184809; US20110105475; U.S. Pat. No. 6,984,560; WO2000037077; WO200040965; WO200076523; WO2003015771; WO2003015777; WO2003016280; WO2003016288; WO2003030612; WO2003016288; WO2003080803; WO2003090745; WO2004007521; WO2004048349; WO2004046162; WO2004048349; WO2005082925; WO2005092328; WO2005097097; WO2007076260; WO2007092751; WO2007140174; WO2007140183; WO2008002573; WO2008025539; WO2008025540; WO200802573; WO2008051942; WO2008073825; WO2008157270; WO2009005998; WO2009012125; WO2009027264; WO2009080555; WO2009127321; WO2009149795; WO2010028981; WO2010034649; WO2010034657; WO2017218330; WO2017218379; WO2017201155; WO2017201152; WO2017201150; WO2017189652; WO2017189651; WO2017189663; WO2017147137; WO2017147159; WO2017147174; WO2017145031; WO2017145040; WO2017145041; WO2017133521; WO2017129125; WO2017128896; WO2017118294; WO2017049172; WO2017049176; WO2017049173; WO2017049177; WO2016173397; WO2016173493; WO2016168553; WO2016161003; WO2016149111; WO2016131414; WO2016130809; WO2016097933; WO2016096115; WO2016096116; WO2016086115; WO2016073767; WO2015138986; WO2018152171; WO2018170165, WO2018170166, WO2018170173, WO2018170182, WO2018170167; WO2017078928; WO2014184271; WO2013007387; WO2012087519; WO2011020615; WO2010069604; WO2013037482; US2017275256; WO2005080064; WO2018190643; WO2018215070; WO2018215610; WO2018214959; WO2018081285; WO2018067704; WO2019007418; WO2018059314; WO2017218337; WO2020231917; WO2020211872; WO2020168143; WO2020168148; WO2020156241; WO2020150136; WO2020114307; WO2020061118; WO2020061114; WO2020061112; WO2020061113; WO2020061116, WO2020061117; WO2020011146; WO2020001304; WO2019160813; WO2019120088; WO2019118571; WO2019089667; WO2019089672; WO2019089665; WO2019089664; WO2019089670; the disclosure of which being incorporated herein by reference.

In an aspect, the FXR agonist can be any FXR agonists disclosed in the following patent applications: WO2017/049172, WO2017/049176, WO2017/049173, WO2017/049177, WO2018/170165, WO2018/170166, WO2018/170173, WO2018/170182, and WO2018/170167.

Specific examples of FXR agonists include but are not limited to EYP001 (Vonafexor), GW4064 (as disclosed in PCT Publication No. WO 00/37077 or in US2007/0015796), 6-ethyl-chenodeoxycholic acids, especially 3α, 7α-dihydroxy 7α-dihydroxy-6α-ethyl-5β-cholan-24-oic acid, also referred to as INT-747 (OCA); INT-777; 6-ethyl-ursodeoxycholic acids, INT-1103, UPF-987, WAY-362450, MFA-1, GW9662, T0901317, fexaramine, 3β-azido-6α-ethyl-7α-hydroxy-5β-cholan-24-oic acid, GS-9674 (Cilofexor) (Phenex Pharmaceuticals AG), Tropifexor (UN452), LMB763 (Nidufexor), PX-102 (PX-20606), PX-104 (Phenex 104), EDP-297, EDP-305, TERN-101 (LY2562175), MET-409, MET-642, WAY362450, Fexaramine, in particular fexaramine-3 (Fex-3), AGN-242266 (former AKN-083, Allergan), BAR502, BAR704, PX20606, PX20350, 3α,7α,11β-Trihydroxy-6α-ethyl-5β-cholan-24-oic Acid (TC-100), 6-(4-{[5-Cyclopropyl-3-(2,6-dichlorophenyl)isoxazol-4-yl]methoxy}piperidin-1-yl)-1-methyl-1H-indole-3-carboxylic Acid, 3,6-dimethyl-1-(2-methylphenyl)-4-(4-phenoxyphenyl)-4,8-dihydro-1H-pyrazolo[3,4-e][1,4]thiazepin-7-one; obeticholic acid, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid, GSK-8062 (CAS No. 943549-47-1). In some embodiments, the FXR agonist is selected from natural bile acids, preferably chenodeoxycholic acid [CDCA] or taurine- or glycine-conjugated CDCA [tauro-CDCA or glyco-CDCA] and synthetic derivatives of natural bile acids, preferably 6-Ethyl-CDCA or taurine- or glycine-conjugated 6-Ethyl-CDCA, natural non-steroidal agonists, preferably Diterpenoids such as Cafestol and Kahweol, or synthetic non-steroidal FXR agonists.

In some embodiments, the FXR agonist is selected from the group consisting of obeticholic acid (Intercept Pharma), cholic acid (CT-RS); GS-9674 (Cilofexor) (Phenex Pharmaceuticals AG), Tropifexor (UN452) (Novartis Pharmaceuticals), LMB763 (Nidufexor), PX-102 (PX-20606), PX-104 (Phenex 104), EYP001, OCA, EDP-297, EDP-305, a steroidal non-carboxylic acid FXR agonist (Enanta Pharmaceuticals), Turofexorate Isopropyl (Pfizer), INT-767 (Intercept Pharmaceuticals), LY-2562175 (Lilly), AGN-242266 (former AKN-083, Allergan), EP-024297 (Enanta Pharmaceuticals), M-480 (Metacrine), TERN-101 (LY2562175), MET-409 (Metacrine), MET-642 (Metacrine), BAR502, RDX-023 (Ardelyx), GW4064, GW6046, WAY362450, Cafestol, Fexaramine and the compound PXL007 (also named EYP001 or EYP001α) identified by the CAS No. 1192171-69-9 (described in WO 2009127321). In a particular embodiment, the FXR agonist is selected from the group consisting of INT-747, the compound identified by EDP-305 a steroidal non-carboxylic acid FXR agonist (Enanta Pharmaceuticals) and the compound identified by the CAS No. 1192171-69-9 (described in WO 2009127321).

In a particular aspect, the FXR agonist is selected from the group consisting of LJN452 (Tropifexor), GS-9674 (Cilofexor), LMB763 (Nidufexor), PX-102 (PX-20606), PX-104 (Phenex 104), OCA (Ocaliva), EDP-297, EDP-305, TERN-101, MET-409, MET-642, GW4064, WAY362450 (Turofexorate isopropyl), Fexaramine, AGN242266 (AKN-083), BAR502 and PXL007 (also named EYP00l).

In a very particular aspect, the FXR agonist is selected from the group consisting of OCA (Ocaliva) (intercept), EDP-297 (Enanta), EDP-305 (Enanta), GS-9674 (Cilofexor) (Gilead), TERN-101 (TERNS), MET-409 (Metacrine), MET-642 (Metacrine), LJN452 (Tropifexor) (Novartis), LMB763 (Nidufexor) (Novartis), and AGN242266 (AKN-083) (Abbvie).

In a particular aspect, the FXR agonist is selected from the group consisting of the compound disclosed in Table 1.

TABLE 1

| | |
|---|---|
| LJN452 (Tropifexor) Cas Number 1383816-29-2 2-(3-((5-cyclopropyl-3-(2- (trifluoromethoxy)phenyl)isoxazol-4- yl)methoxy)-8-azabicyclo[3.2.1]octan-8-yl)-4- fluorobenzo[d]thiazole-6-carboxylic acid | 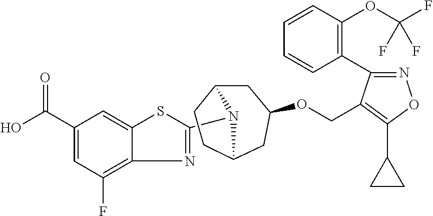 [SMILES: O=C(O)c1cc(F)c2nc(N3[C@@H]4CC[C@H]3C[C@@H](OCc3c(-c5ccccc5OC(F)(F)F)noc3C3CC3)C4)sc2c1] |
| LMB763 (Nidufexor) Cas Number 1773489-72-7 4-[(N-benzyl-8-chloro-1-methyl-1,4- dihydro[1]benzopyrano[4,3-c]pyrazole-3- carboxamido)methyl]benzoic acid | 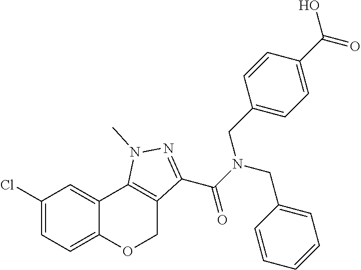 [SMILES: Cn1nc(C(=O)N(Cc2ccccc2)Cc2ccc(C(=O)O)cc2)c2c1-c1cc(Cl)ccc1OC2] |
| GS-9674 (Cilofexor) Cas Number 1418274-28-8 2-[3-[2-Chloro-4-[[5-cyclopropyl-3-(2,6- dichlorophenyl)-4- isoxazolyl]methoxy]phenyl]-3-hydroxy-1- azetidinyl]-4-pyridinecarboxylic acid | 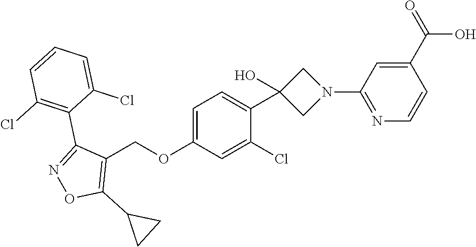 [SMILES: O=C(O)c1ccnc(N2CC(O)(c3ccc(OCc4c(-c5c(Cl)cccc5Cl)noc4C4CC4)cc3Cl)C2)c1] |
| PX-102 (PX-20606) Cas Number 1268244-85-4 4-(2-(2-Chloro-4-((5-cyclopropyl-3-(2,6- dichlorophenyl)isoxazol-4- yl)methoxy)phenyl)cyclopropyl)benzoic acid | 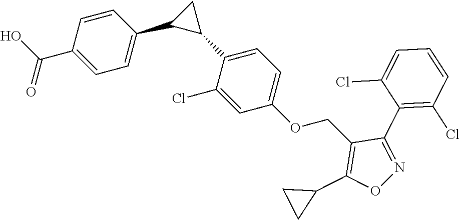 |
| PX-104 or Phenex 104 | enantiomer of PX-102 |
| OCA (Ocaliva or INT-747) Cas Number 459789-99-2 Cholan-24-oic acid, 6-ethyl-3,7-dihydroxy-, (3α,5β,6α,7α)- | 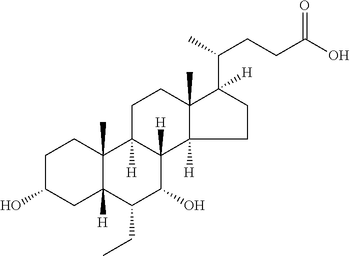 |
| EDP-305 Cas Number 1933507-63-1 Benzenesulfonamide, 4-(1,1-dimethylethyl)- N-[[[(3α,5β,6α,7α)-6-ethyl-3,7-dihydroxy-24- norcholan-23-yl]amino]carbonyl]- | 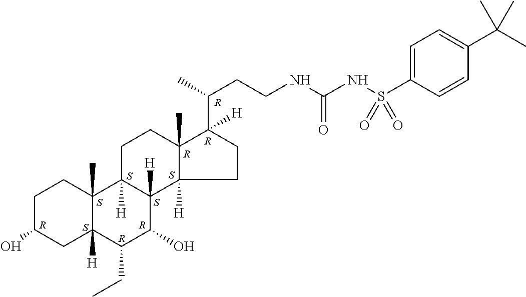 |
| TERN-101 (LY2562175) Cas Number 1103500-20-4 6-(4-{[5-Cyclopropyl-3-(2,6- dichlorophenyl)isoxazol-4- yl]methoxy}piperidin-1-yl)-1-methyl-1H- indole-3-carboxylic acid | 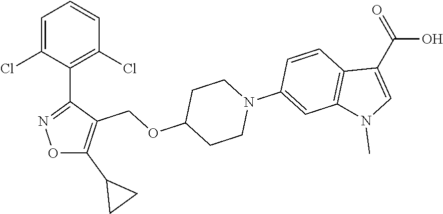 [SMILES: Cn1cc(C(=O)O)c2ccc(N3CCC(OCc4c(-c5c(Cl)cccc5Cl)noc4C4CC4)CC3)cc21] |
| MET409 | Developed by Metacrine |
| MET642 | Disclosed in WO2017049173 |
| | 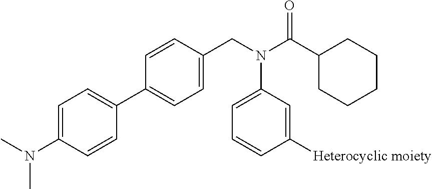 |
| GW4064 Cas Number 278779-30-9 3-[2-[2-Chloro-4-[[3-(2,6-dichlorophenyl)-5- (1-methylethyl)-4- isoxazolyl]methoxy]phenyl]ethenyl]benzoic acid | 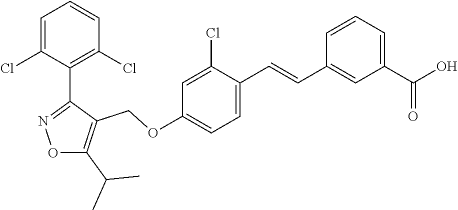 [SMILES: CC(C)c1onc(-c2c(Cl)cccc2Cl)c1COc1ccc(/C=C/c2cccc(C(=O)O)c2)c(Cl)c1] |
| WAY362450 (Turofexorate isopropyl or XL335 or FXR450) Cas Number 629664-81-9 3-(3,4-Difluoro-benzoyl)-1,1-dimethylene- 1,2,3,6-tetrahydro-azepino [4,5-b]indole-5- carboxylic acid isopropyl ester, 3-(3,4- Difluorobenzoyl)-1,2,3,6-tetrahydro-1,1- dimethyl-azepino[4,5-b]indole-5-carboxylic acid 1-methylethyl ester, | 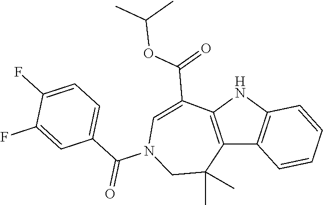 [SMILES: CC(C)OC(=O)C1=CN(C(=O)c2ccc(F)c(F)c2)CC(C)(C)c2c1[nH]c1ccccc21] |
| Fexaramine Cas Number 574013-66-4 3-[3-[(Cyclohexylcarbonyl)[4′- (dimethylamino) [1,1′-biphenyl]-4- yl]methyl]amino]phenyl]-2-propenoic acid methyl ester | 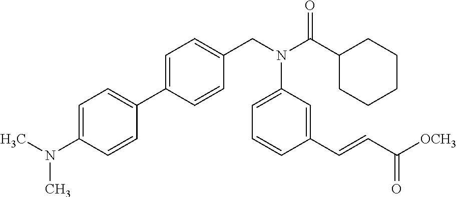 [SMILES: COC(=O)/C=C/c1cccc(N(Cc2ccc(-c3ccc(N(C)C)cc3)cc2)C(=O)C2CCCCC2)c1] |
| AGN242266 (AKN-083) | 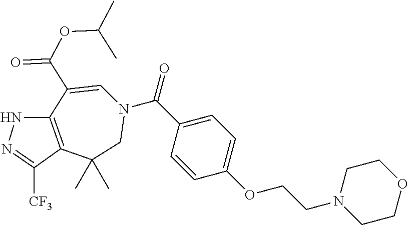 [SMILES: CC(C)OC(=O)C1=CN(C(=O)c2ccc(OCCN3CCOCC3)cc2)CC(C)(C)c2c(C(F)(F)F)n[nH]c21] |
| BAR502 Cas Number 1612191-86-2 6a-ethyl-3α, 7α-dihydroxy-24-nor-5β-cholan- 23-ol | 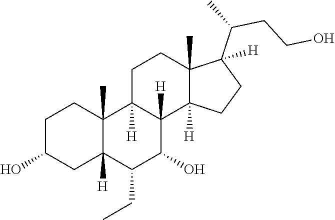 |
| EYP001 (Vonafexor) Cas Number 1192171-69-9 | 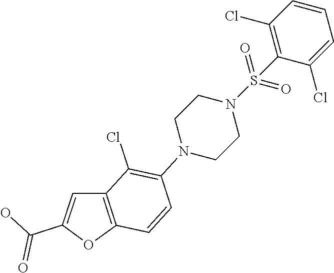 [SMILES: O=C(O)c1cc2c(Cl)c(N3CCN(S(=O)(=O)c4c(Cl)cccc4Cl)CC3)ccc2o1] |

and any pharmaceutically acceptable salt thereof.

The FXR agonist can be administered once, twice or three times a day, preferably once or twice, for example in the morning (e.g., between 6 and 10 am) or in the evening (e.g., 6 and 10 pm). In one aspect, the FXR agonist is administered once a day. In another aspect, the FXR agonist is administered twice a day. It is preferably administered every day. However, an administration every 2, 3, 4, 5, 6 or 7 days can also be contemplated. The daily dosage of the FXR agonist may be varied over a wide range from 1 μg to 1,000 mg per adult per day. The FXR agonist can be administered by oral, sublingual, subcutaneous, intramuscular, intravenous, transdermal, intratumoral, local or rectal administration, preferably for oral administration.

### TLR3 Agonist

The TLR3 agonists according to the present invention can be selected from any suitable agent. For example, TLR3 agonists can be selected from a range of nucleic acid agonists; other agonists can be tested using known assays.

Generally, any proteinaceous, nucleic acid or small molecule candidate TLR3 agonist can be identified using known assays. For example, assays for detecting TLR3 agonism of test compounds are described, for example, in PCT publication nos. WO 03/31573, WO 04/053057, WO 04/053452, and WO 04/094671, the disclosures of each of which are incorporated herein by reference.

Regardless of the particular assay employed, a compound can be identified as an agonist of TLR3 if performing the assay with the compound results in an increase of some biological activity mediated by TLR3. Unless otherwise indicated, an increase in biological activity refers to an increase in the same biological activity over that observed in an appropriate control. For instance, a TLR3 activity can be increased by at least 10, 20, 30, 40, 50, 60, 70, 80, 90, 100, 200 or 300% when compared to the TLR3 activity in absence of the compound.

In certain aspects, the TLR3 agonist can be a natural agonist of TLR3 or a synthetic TLR3 agonist. TLR3 agonists are well known in the art and suitable TLR3 agonists are available. Further TLR3 agonists, or derivatives or analogs of known TLR3 agonists can be readily identified, made and/or assessed. For instance, TLR3 agonists are disclosed in the following reviews (Le Naour et al, Oncoimmunology, 2020, 9, 1-13).

The most commonly used TLR3 agonists are nucleic acid based agonists. Thus in preferred aspects, TLR3 agonists are nucleotide or nucleic-acid based. Nucleotide or nucleic-acid based compounds can be assessed for their ability to act as a TLR3 agonist using readily available methods. The nucleic acid based TLR3 agonist can be single-stranded or double-stranded or a mixture thereof. The nucleic acid based TLR3 agonist can comprise deoxyribonucleotides, or ribonucleotides or a mixture thereof. The nucleotides can be natural or synthetic, and may be derivatives or analogs of natural nucleotides, such as for example in Kandimalla et al. ((2003) Nucl. Acid. Res. 31(9): 2393-2400). In an aspect, the TLR3 agonist has no or low homology (e.g., less than 10, 20, 30, 40%) with the subject genome, in particular with human genome.

The particular TLR3 agonist can be a double stranded RNA compound (dsRNA) referred to as polyadenylic-polyuridylic acid, i.e., poly(A):poly(U), pApU, polyAU or polyA:U each of these terms being equivalent. PolyAU is generally an at least partially double stranded molecule made of polyadenylic acid(s) and polyuridylic acid(s), each optionally substituted with other monomers so long as the biological function (e.g. TLR3 agonism) is preserved.

Within the context of the present invention, the term “double-stranded RNA” molecule designates any therapeutically or prophylactically effective (synthetic) double-stranded RNA compound. Such compounds are typically active per se, i.e., they do not encode a polypeptide or do not require translation to be active. dsRNA TLR3 agonists can have any suitable length. Preferably, a dsRNA TLR3 agonist has a length of at least about 10 base pairs (bp), 20 bp, 30 bp, 50 bp, 80 bp, 100 bp, 200 bp, 400 bp, 600 bp, 800 bp or 1000 bp. In one aspect, the dsRNA molecule is a short dsRNA having a chain length of less than 30 bp, 50 bp, 80 bp, 100 bp or 200 bp. In another aspect, the dsRNA molecule is a longer dsRNA, but having a chain length of less than 400 bp, 600 bp, 800 bp or 1000 bp. In another aspect, the dsRNA molecule is a long dsRNA having a chain length of greater than 1000 bp.

In one aspect, a dsRNA composition comprises a heterogenous mixture of dsRNA molecules, wherein a plurality of molecules have differing lengths. Preferably the dsRNA molecules have on average a length of at least about 10 bp, 20 bp, 30 bp, 50 bp, 80 bp, 100 bp, 200 bp, 400 bp, 600 bp, 800 bp or 1000 bp. In another embodiment, a dsRNA composition comprises a plurality dsRNA molecules where at least 20%, 50%, 80%, 90% or 98% of dsRNA molecules have a length of at least about 10 bp, 20 bp, 30 bp, 50 bp, 80 bp, 100 bp, 200 bp, 400 bp, 600 bp, 800 bp or 1000 bp. In a preferred aspect, dsRNA composition has a substantially homogenous mixture of dsRNA molecules, where substantially all the molecules do not differ in chain length by more than 30 bp, 50 bp, 80 bp, 100 bp or 200 bp. Average chain length of nucleic acid TLR3 agonists can be determined easily, for example, by gel permeation chromatography.

In one aspect, the dsRNA composition comprises a heterogenous mixture of dsRNA molecules having a length in the range from 100, 200, 300, 400, 500, 600, 700, 800, 900, 1000, 1100, 1200, 1300, 1400 or 1500 bp to 1500, 2000, 3000, 4000, 5000, 6000, 7000, 8000, 9000 or 10,000 bp. In a more specific aspect, the dsRNA composition comprises a heterogenous mixture of dsRNA molecules having a length in the range from 100, 200, 300, 400, or 500 bp to 700, 800, 900, 1000, 1100, 1200, 1300, 1400 or 1500 bp, for instance from 100, 200 or 300 bp to 900, 1000 or 1100 bp. In another more specific aspect, the dsRNA composition comprises a heterogenous mixture of dsRNA molecules having a length in the range from 1300, 1400, 1500, 1600 or 1700 bp to 6000, 7000, 8000, 9000 or 10,000 bp, for instance from 1400, 1500 or 1600 bp to 7000, 8000 or 9000 bp.

Previous studies of double-stranded RNA (dsRNA) assessing their ability to be effective interferon inducers suggested that dsRNA agents must possess the secondary structure of a double stranded helix. Other dsRNA agents which have also been shown to be suitable as TLR3 agonist include double-stranded polynucleotides which are not complementary or not perfectly complementary; these have been known as, so-called “mismatched” or “loop-out” structures and exist in naturally occurring RNAs such as transfer tRNAs, ribosomal RNAs and the viral RNA secondary structures. One commonly cited dsRNA compound, Ampligen, comprises a structure where few parts of cytidine in the poly L:poly C (also named poly(I):poly(C), plpC, polylC or polyl:C) structure are replaced with uridine (i.e. mismatched RNA); this compound has been reported to have physiological activity similar to that of the parent polyl:C. However, it will be appreciated that TLR3 agonists of any type and configuration can be used in accordance with this invention.

In a particular aspect, each strand of these dsRNAs can have a length comprised between about 5 and 50 bases, more preferably between 5 and 40, 35, 30, 25 or 20 bases. Each strand is preferably perfectly complementary to the other. Preferred examples of such dsRNAs are homopolyRNAs, i.e., dsRNAs in which each strand consists essentially of a repeat of the same base; or comprise a homopolyRNA region.

The base may be any naturally occurring base (e.g., polyA, polyU, polyC, polyG) or non-naturally occurring (e.g., chemically synthesized or modified) base (e.g., polyl). Polynucleotides typified by polyinosinic-polycytidylic acid, i.e., poly(I):poly(C), plpC or polyl:C and polyadenylic-polyuridylic acid, i.e., poly(A):poly(U), pApU or polyA:U, are well-known compounds in the art and have been known to induce interferon production by immune cells. Thus in preferred aspects, the TLR3 agonist is a double stranded nucleic acid selected from the group consisting of: polyinosinic acid and polycytidylic acid, polyadenylic acid and polyuridylic acid, polyinosinic acid analog and polycytidylic acid, polyinosinic acid and polycytidylic acid analog, polyinosinic acid analog and polycytidylic acid analog, polyadenylic acid analog and polyuridylic acid, polyadenylic acid and polyuridylic acid analog, and polyadenylic acid analog and polyuridylic acid analog.

In a particular aspect, the TLR3 agonist is a Poly I:C (polyribosinic:polyribocytidic acid) having a length in the range from 100, 200, 300, 400, 500, 600, 700, 800, 900, 1000, 1100, 1200, 1300, 1400 or 1500 bp to 1500, 2000, 3000, 4000, 5000, 6000, 7000, 8000, 9000 or 10,000 bp. In a more specific aspect, the TLR3 agonist is a Poly I:C (polyribosinic:polyribocytidic acid) having a length in the range from 100, 200, 300, 400, or 500 bp to 700, 800, 900, 1000, 1100, 1200, 1300, 1400 or 1500 bp, for instance from 100, 200 or 300 bp to 900, 1000 or 1100 bp. In another more specific aspect, the TLR3 agonist is a Poly I:C (polyribosinic:polyribocytidic acid) having a length in the range from 1300, 1400, 1500, 1600 or 1700 bp to 6000, 7000, 8000, 9000 or 10,000 bp, for instance from 1400, 1500 or 1600 bp to 7000, 8000 or 9000 bp.

It will be appreciated that nucleic acid-based agonists of TLR3 can be designed using any suitable method. Preferably, the basic requirement of stability and resistance to nuclease attack and the preferences for chain length are taken into account, and that structural changes can be tested and assessed with reference to the a rAn:rUn or rln:rCn complex for example. Measures can be taken to increase stability and resistance to nucleases, or to increase or optionally decrease interferon-inducing action.

Other examples of dsRNA include nucleic acids described in U.S. Pat. Nos. 5,298,614 and 6,780,429. U.S. Pat. No. 5,298,614 reports that when chain length of the double stranded nucleic acid derivatives is limited to certain ranges, the resulting substances exhibit desired physiological activity with markedly less toxicity, providing polynucleotides having a length of about 50 to 10,000 as calculated by base pair numbers. Also described are derivative wherein the purine or pyrimidine ring in the nucleic acid polymer is substituted with at least one SH group, or said derivative contains a disulphide bond, or both (preferred ratio of number of sulphur atoms to cytidylic acid present in the poly C are 1:6 to 39). U.S. Pat. No. 6,780,429 describes a particular type of dsRNA compounds that are “chain-shortened” having lengths of about 100 to 1,000 as calculated by base pair numbers, or preferably from 200 to 800, and more preferably from 300 to 600. The latter compounds are reported to contain low numbers of 2′-5′ phosphodiester bonds by a method designed to avoid phosphate groups causing intramolecular rearrangement from 3′ position to 2′ position through a mechanism called pseudo rotation simultaneously that can occur during hydrolysis of poylnucleotides, resulting in a portion of 3′-5′ phosphodiester bonds in the chain-shortened polynucleotide molecule being replaced by 2′-5′ phosphodiester bonds. The disclosures of each of these references are incorporated herein by reference.

Other nucleic acid agonists that can be suitable for use as TLR3 agonists are provided in: Field et al: Proc. Nat. Acad. Sci. U.S. 58, 1004, (1967); Field et al: Proc. Nat. Acad. Sci. U.S. 58, 2102, (1967); Field et al: Proc. Nat. Acad. Sci. U.S. 61, 340, (1968); Tytell et al: Proc. Nat. Acad. Sci. U.S. 58, 1719, (1967); Field et al: J. Gen. Physiol. 56, 905 (1970); De Clercq et al: Methods in Enzymology, 78, 291 (1981). A number of synthetic nucleic acid derivatives have been described, including homopolymer-homopolymer complexes (Double Strand Nucleic Acid Polymer such as those in which polyl:C or polyA:U are a parent structure, where these homopolymer-homopolymer complexes contain: (1) base modifications, exemplified by polyinosinic acid-poly(5-bromocytidylic acid), polyinosinic acid-poly(2-thiocytidylic acid), poly(7-deazainosinic acid)-polycytidylic acid, poly(7-deazainosinic acid)-poly(5-bromocytidylic acid), and polyinosinic acid-poly(5-thiouridylic acid); (2) Sugar Modifications, exemplified by poly(2′-azidoinosinic acid)-polycytidylic acid; and (3) phosphoric Acid Modifications, exemplified by polyinosinic acid-poly(cytidyl-5′-thiophosphoric acid). Other synthetic nucleic acid derivatives that have been described include interchanged copolymers, exemplified by poly(adenylic acid-uridylic acid) or polyA:U; and homopolymer-copolymer complexes, exemplified by polyinosinic acid-poly(cytidylic acid-uridylic acid) or polyl:C and polyinosinic acid-poly(citydylic acid-4-thiouridylic acid). Other synthetic nucleic acid derivatives that have been described include complexes of synthetic nucleic acid with polycation, exemplified by polyinosinic acid-polycytidylic acid-poly-L-lysinecarboxy-methylcellulose complex (called “Poly ICLC”). Yet another example of synthetic nucleic acid derivative is polyinosinic acid-poly(1-vinylcytosine).

One example of a TLR3 agonist is Ampligen™ (Hemispherx, Inc., of Rockville, Md., U.S.A.), a dsRNA formed by complexes of polyriboinosinic and polyribocytidylic/uridylic acid, such as rln:r(Cx,U or G)n where x has a value from 4 to 29, e.g., rln:r(C12 U)n. Many mismatched dsRNA polymers which behave similarly to Ampligen™ have been studied; mismatched dsRNA based on polyl:C have included complexes of a polyinosinate and a polycytidylate containing a proportion of uracil bases or guanidine bases, e.g., from 1 in 5 to 1 in 30 such bases. The key therapeutic advantage of mismatched dsRNAs over other forms of natural and/or synthetic dsRNAs a reported reduction in toxicity over compounds such as those described in Lampson et al in U.S. Pat. No. 3,666,646.

Specific examples of double-stranded RNA according to the present invention further include Polyadenur™ (Ipsen) and Ampligen™ (Hemispherx). Polyadenur™ is a polyA:U RNA molecule, i.e., contains a polyA strand and a polyU strand. Polyadenur™ has been developed for the potential treatment of hepatitis B virus (HBV) infection. Ampligen™ is of a poly(I):poly(C) compound (or a variant thereof comprising a poly(I):poly(C12U) RNA molecule). Ampligen is disclosed for instance in EP 281380 or EP 113 162. Ampligen™ has been proposed for the treatment of cancer, viral infections and immune disorders. It was developed primarily for the potential treatment of myalgic encephalomyelitis (ME, or chronic fatigue syndrome/chronic fatigue immune dysfunction syndrome, CFS/CFIDS). Ampligen™ is also known as AMP-516 or Rintatolimod.

Other TLR3 agonists can also be cited as example, including:
Poly I:C (polyribosinic:polyribocytidic acid) formulated for delivery with polyethylenimine (BO-112);
PolyICLC (Hiltonol or polyriboinosinic-polyribocytidylic acidpolylysine carboxymethylcellulose, also known as Hiltonol) (Salazar et al., Cancer Immunol Res, 2014, 2, 720-724; Levy et al., Journal of Infectious Diseases, 1975, 132, 434-439);
Riboxxol (RGIC®50) Riboxxon (Riboxx Pharmaceuticals) (Naumann et al, Clin Dev Immunol., 2013, 2013, 283649);
RIBOXXIM or RGC100 (Naumann et al., Clinical and Developmental Immunology, 2013, 2013, 283649) (Riboxx Pharmaceuticals);
APOXXIM (Riboxx Pharmaceuticals);
TL-532 (Tollys; https://tollys.fr/tl-532/) and related molecules as disclosed in WO2019211492, the disclosure of which being incorporated herein by reference;

| | |
|---|---|
| 532 | Sense 70 bases: 10 I-50 A-10 I |
| | Antisense 70 bases: 10 C-50 U-10 C |

ARNAX (Matsumoto et al, Nat Commun, 2015, 6, 6280); or
MCT-465 and MCT-485.

Additional TLR3 agonists are described in the following patent applications: WO09130616, WO09105260, WO09102496, WO08106803, WO08109083, the disclosure of which being incorporated herein by reference.

Small molecules having TLR3 agonist activity are also known by the person skilled in the art. For instance, Zhang et al (2017, J. Med. Chem. 2017, 60, 5029-5044) discloses several TLR3 agonists. Some of them are the following:

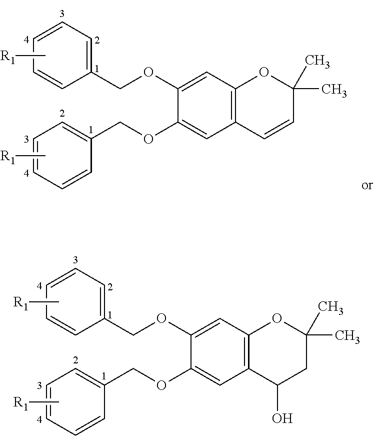

with R1 being 4-NO₂;

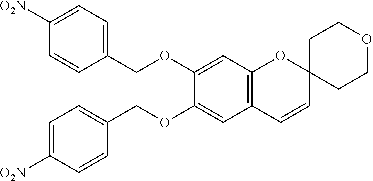 [SMILES: O=[N+]([O-])c1ccc(COc2cc3c(cc2OCc2ccc([N+](=O)[O-])cc2)OC2(C=C3)CCOCC2)cc1]

In a preferred aspect, the TLR3 agonist is Rintatolimod, Hiltonol or Riboxxol.

The TLR3 agonist can be administered by oral, sublingual, subcutaneous, intramuscular, intravenous, transdermal, intratumoral, local or rectal administration, preferably for intravenous administration.

### Preferred Combinations

In very particular aspects, the TLR3 agonist and the FXR agonist are selected as following:
the FXR agonist is selected from the group consisting of EYP001 (Vonafexor), LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502;
the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva);
the FXR agonist is EYP001 (Vonafexor);
the TLR3 agonist is selected in the group consisting of Poly I:C (polyribosinic:polyribocytidic acid), polyA:U (poly(adenylic acid-uridylic acid), Poly ICLC (polyinosinic acid-polycytidylic acid-poly-L-lysinecarboxy-methylcellulose complex or Hiltonol), Polyl:polyC₁₂U (polylC₁₂U, Ampligen or Rintatolimod), Riboxxol (RGIC®50), RIBOXXIM (RGIC®100), APOXXIM, TL-532, ARNAX, IPH3102, MCT-465 and MCT-485;
the TLR3 agonist is a Poly I:C (polyribosinic:polyribocytidic acid);
the TLR3 agonist is selected from the group consisting of Riboxxol, Rintatolimod, and Hiltonol;
the TLR3 agonist is Riboxxol;
the FXR agonist is selected from the group consisting of EYP001 (Vonafexor), LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502; and the TLR3 agonist is selected in the group consisting of Poly I:C (polyribosinic:polyribocytidic acid), polyA:U (poly(adenylic acid-uridylic acid), Poly ICLC (polyinosinic acid-polycytidylic acid-poly-L-lysinecarboxy-methylcellulose complex or Hiltonol), Polyl:polyC₁₂U (polylC₁₂U, Ampligen or Rintatolimod), Riboxxol (RGIC®50), RIBOXXIM (RGIC®100), APOXXIM, TL-532, ARNAX, IPH3102, MCT-465 and MCT-485;
the FXR agonist is selected from the group consisting of EYP001 (Vonafexor), LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502; and the TLR3 agonist is a Poly I:C (polyribosinic:polyribocytidic acid);
the FXR agonist is selected from the group consisting of EYP001 (Vonafexor), LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502; and the TLR3 agonist is selected from the group consisting of Riboxxol, Rintatolimod, and Hiltonol;
the FXR agonist is selected from the group consisting of EYP001 (Vonafexor), LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502; and the TLR3 agonist is Riboxxol;
the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva); and the TLR3 agonist is selected in the group consisting of Poly I:C (polyribosinic:polyribocytidic acid), polyA:U (poly(adenylic acid-uridylic acid), Poly ICLC (polyinosinic acid-polycytidylic acid-poly-L-lysinecarboxy-methylcellulose complex or Hiltonol), Polyl:polyC₁₂U (polylC₁₂U, Ampligen or Rintatolimod), Riboxxol (RGIC®50), RIBOXXIM (RGIC®100), APOXXIM, TL-532, ARNAX, IPH3102, MCT-465 and MCT-485;
the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva); and the TLR3 agonist is a Poly I:C (polyribosinic:polyribocytidic acid);
the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva); and the TLR3 agonist is selected from the group consisting of Riboxxol, Rintatolimod, and Hiltonol;
the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva); and the TLR3 agonist is Riboxxol;
the FXR agonist is EYP001 (Vonafexor); and the TLR3 agonist is selected in the group consisting of Poly I:C (polyribosinic:polyribocytidic acid), polyA:U (poly(adenylic acid-uridylic acid), Poly ICLC (polyinosinic acid-polycytidylic acid-poly-L-lysinecarboxy-methylcellulose complex or Hiltonol), Polyl:polyC₁₂U (polylC₁₂U, Ampligen or Rintatolimod), Riboxxol (RGIC®50), RIBOXXIM (RGIC®100), APOXXIM, TL-532, ARNAX, IPH3102, MCT-465 and MCT-485;
the FXR agonist is EYP001 (Vonafexor); and the TLR3 agonist is a Poly I:C (polyribosinic:polyribocytidic acid);
the FXR agonist is EYP001 (Vonafexor); and the TLR3 agonist is selected from the group consisting of Riboxxol, Rintatolimod, and Hiltonol;
the FXR agonist is EYP001 (Vonafexor); and the TLR3 agonist is Riboxxol.

In a particular aspect, the FXR agonist is to be administered by oral route whereas the TLR3 agonist is to be administered by another route, such as an intravenous administration.

### Therapeutic Uses

The combination of the TLR3 agonist and the FXR agonist can be used for the treatment of any disease or disorder that can have a benefit of the activation of the innate immunity and/or an increased production of pro-inflammatory cytokines including type I interferon (IFN).

The combination of the TLR3 agonist and the FXR agonist can be used as vaccine adjuvant. Accordingly, the present invention relates to a vaccine composition comprising a TLR3 agonist and an FXR agonist. The vaccine may comprise one or several antigens or nucleic acids encoding said antigens. The antigen can be a viral, bacterial or tumor associated antigen depending on the therapeutic use of the vaccine.

The disease can be non-exhaustively selected from the group consisting to an infection, especially a viral infection, a bacterial infection or a protozoan infection, a cancer, and an autoimmune disease.

In one aspect, the disease is an infection by a virus, especially an hepatotropic virus. This virus can be hepatitis A virus, hepatitis B virus, hepatitis C virus, hepatitis D virus, or hepatitis E virus, preferably hepatitis B virus, or hepatitis D virus, more preferably hepatitis B virus.

The virus can alternatively be selected from the group consisting of Herpesviridae virus including herpes simplex virus (HSV), varicella-zoster virus, Kaposis sarcoma herpesvirus and cytomegalovirus (CMV), Hepadnaviridae virus including HBV, papillomavirus (HPV), coronavirus including SARS-Cov1, MERS-Cov and SARS-Cov2, retrovirus including HIV, influenza virus and rhinoviruses. In a preferred aspect, the disease is an infection by a coronavirus, especially SARS-Cov 2 infection, more particularly cognitive impairment and fatigue associated with SARS-Cov 2 infection. In another specific aspect, the disease is a HIV infection, in particular a chronic HIV infection. In an additional specific aspect, the disease is a HBV infection, in particular a chronic HBV infection.

The disease can be a cancer, particularly a solid cancer or a hematopoietic cancer, preferably chosen among AIDS-related Kaposi's sarcoma, leukemia such as hairy-cell leukemia, chronic myeloid leukemia, and non-Hodgkin's leukemia, lymphoma such as follicular lymphoma, B-cell lymphoma, cutaneous T-cell lymphoma and adult T-cell leukemia-lymphoma, carcinoid tumors, melanoma, multiple myeloma, renal cell carcinoma, colorectal adenocarcinoma, hepatocarcinoma, breast cancer, prostate cancer, ovarian cancer, pancreas cancer, peritoneal cancer, bladder cancer, lung cancer, glioblastoma, oral carcinoma, glioma, head and neck cancer, sarcoma, and neuroendocrine tumors.

The disease can be chronic fatigue syndrome.

The disease could be a bacterial infection, in particular mycobacterial infection. The disease can also be a protozoan infection, in particular leishmaniasis.

The disease can be an autoimmune disease, for instance selected in the non-exhaustive list consisting of multiple sclerosis, rheumatoid arthritis, Behçet's syndrome, Churg-Strauss syndrome, Guillain-Barre syndrome, and inflammatory bowel disease including ulcerative colitis and Crohn's disease.

The disease can be a myoproliferative disorder such as thrombocythemia, polycythemia vera and agnogenic myeloid metaplasia, fibrosis such as cryptogenic fibrosing alveolitis, osteoporosis, and degenerative inflammatory diseases.

In a particular aspect, the disease is selected among a HBV infection, a HDV infection, a chronic fatigue syndrome, and a coronavirus, especially SARS-Cov 2 infection, more particularly associated with cognitive impairment and chronic fatigue.

More particularly, the disease can be a co-infection by HBV and HDV. The patient to be treated can be coinfected and super-infected. The terms “coinfected patients” refers to individuals that have been simultaneously infected with HBV and HDV. The terms “super-infected patients” refers to individuals that have been firstly infected with HBV, and then infected with HDV.

### Additional Active Ingredient(s)

The pharmaceutical composition, product, kit of combined preparation as disclosed herein can further comprise or be used in combination to one or several additional active ingredients. Additional active ingredients can be selected among the active ingredients already known for their use in combination with a TLR3 agonist or a FXR agonist.

The combination of the TLR3 agonist and FXR agonist can be used as vaccine adjuvant and can be used or combined with any vaccine, for instance a vaccine against cancer or a vaccine directed against bacterial, viral or parasitic infection.

In another aspect, when the disease is a viral infection, the additional active ingredient can be an antiviral, more particularly an antiviral having an activity against HBV when the disease is HBV infection. In a preferred aspect, the at least one additional active ingredient is a polymerase inhibitor selected from the group consisting of L-nucleosides, deoxyguanosine analogs and nucleoside phosphonates. In a very specific aspect, the at least one additional active ingredient is selected from the group consisting of lamivudine, telbivudine, emtricitabine, entecavir, adefovir and tenofovir. The ingredient can also be azidothymidine. The additional active ingredient can also be an interferon such as IFNα and a pegylated form thereof, in particular interferon α2α. Alternatively, the additional active ingredient can be an antibody directed against a viral protein. Finally, the additional active ingredient can be a vaccine directed against one or several viruses. When the disease is a HIV infection, the additional active ingredient can be DCVAX-001, a fusion protein containing a human monoclonal antibody specific for the dendritic cell receptor, DEC-205 (CD205), and the HIV gag p24 protein.

In another aspect, when the disease is a cancer, the additional active ingredient can be an antitumor agent, such as a chemotherapeutic antitumor agent such as cyclophosphamide, temozolomide or decitabine, a HDAC inhibitor such as Romidepsin, an antibody or a multispecific molecule derivated therefrom, a peptide vaccine or an immune cell such as a CAR T cell. The antibody can be directed against a tumor associated antigen or an immunomodulating antibody, for instance an antibody directed against OX40, 4-1BB, PD-1, PD-L1, TIM3, CTLA-4, CD27 or CD73. For instance, the peptide vaccine could be IMA950 multipeptide vaccine, MUC1 vaccine, CDX-1401 vaccine, GAA/TT-peptide vaccine, GBM6-AD vaccine or HspE7 vaccine among others. For instance, the antibody could be a PD-1 antagonist such as retifanlimab, pembrolizumab or nivolumab, a PD-L1 antagonist such as atezolizumab or durvalumab, an anti-CTLA-4 antibody such as Tremelimumab or Ipilimumab, a CD27 agonist such as Varlilumab, an antibody targeted a tumor associated antigen such as oregovomab.

In a further aspect, the additional active ingredient can be an anti-inflammatory agent such as a COX-2 inhibitor such as Celecoxib.

When the disease is an auto-immune disease, especially multiple sclerosis and Crohn's disease, the additional active ingredient can be an antibody directed against α4-integrin such as natalizumab.

### Examples

### Materials and Methods

### Primary Human Hepatocytes

Primary human hepatocytes (PHH) were freshly prepared from human liver resection obtained from the Centre Léon Bérard (Lyon) with French ministerial authorizations (AC 2013-1871, DC 2013-1870, AFNOR NF 96 900 sept 2011) as previously described (Lecluyse, E. L. & Alexandre, E. Methods Mol. Biol. Clifton NJ 640, 57-82 (2010)).

### Virus

HBV stocks (genotype D, Genbank ID U95551) were prepared using the HepAD38 cell line according to previously described protocols (Ladner, S. K. et al. Antimicrob. Agents Chemother. 41, 1715-1720 (1997)). Supernatants containing HBV particles were clarified (0.45 μm filter) and concentrated with 8% PEG 8000 (Sigma-Aldrich). HBV DNA was quantified using the AmpliPrep/COBAS® TaqMan® HBV Test (Roche).

### Chemicals

EYP-001 was provided by Enyo Pharma. A stock at 30 mM was constituted in DMSO, and working concentrations were obtained by dilution into the culture medium extemporaneously. Riboxxol was purchased from Riboxx (Germany). It is a synthetic double-stranded RNA (dsRNA), has a length of 50 bp, and is composed of cytosines, inosines and guanosines. It is a pure agonist of TLR3, with no leakiness toward RIG-1 or MDA5 (Lucifora J et al. Sci Rep. 2018 Mar. 29; 8(1):5390). A stock solution was constituted at 1 mg/mL in RNAse-free water. Poly(I:C) High and Low molecular weight were purchased from InvivoGen (USA). They are both double stranded RNA composed of inosine and cytosine (Low: 0.2 to 1 kb, High: 1.5 to 8 kb) and are TLR3 agonists. A stock solution at 1 mg/mL was constituted in RNAse-free water. Tropifexor, Cilofexor and Nidufexor were purchased from TargetMol (USA), GW4064 from Selleckchem (USA), Fexaramine and OCA from Euromedex (France). Those 6 FXR agonists were used as described below. All the stock solutions were at 10 mM (Cilofexor, Tropifexor, Nidufexor, GW4064) or 50 mM (Fexaramine, OCA) in DMSO.

### HBsAg and HBeAg Quantification

HBs and HBe antigens secreted in cells supernatant were quantified, after required dilutions, with Autobio kits (AutoBio, China) according to manufacturer's protocol.

### Results

The combination with an FXR agonist strongly potentiates (synergistic effect) the inhibitory effects of TLR3 agonists on HBV replication in PHH.

To determine the combined impact of FXR agonists and TLR3 agonists on HBV infection, in vitro infections were performed in primary human hepatocytes (PHH). PHH are naturally susceptible to infection with HBV virions produced in vitro, leading to very high levels of replication of the virus. Freshly prepared and seeded PHH were infected with HBV at a multiplicity of infection of 500 vge/cell. From day 4 to 12 post-infection, cells were treated with the FXR agonist EYP001 at 30 μM, or with a TLR3 agonist (Riboxxol) at 5 μg/mL, or with the combination of the FXR agonist EYP001 at 30 μM with a TLR3 agonist (Riboxxol) at 5 μg/mL, or with the vehicle. Supernatants were harvested at day 12 for secreted HBV DNA and secreted antigens (HBsAg and HBeAg) quantification. Results are expressed as the mean+SD of two independent experiments, each performed with at least three biological replicates.

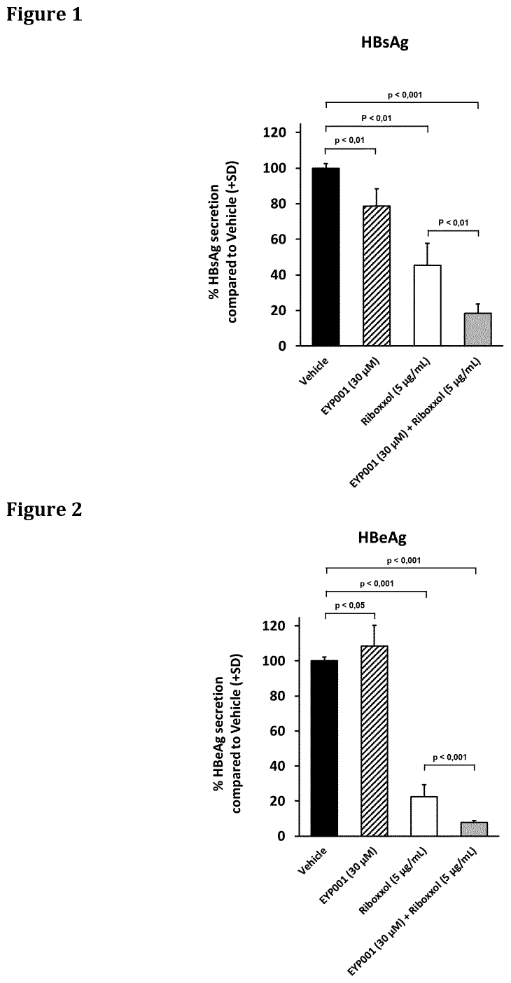

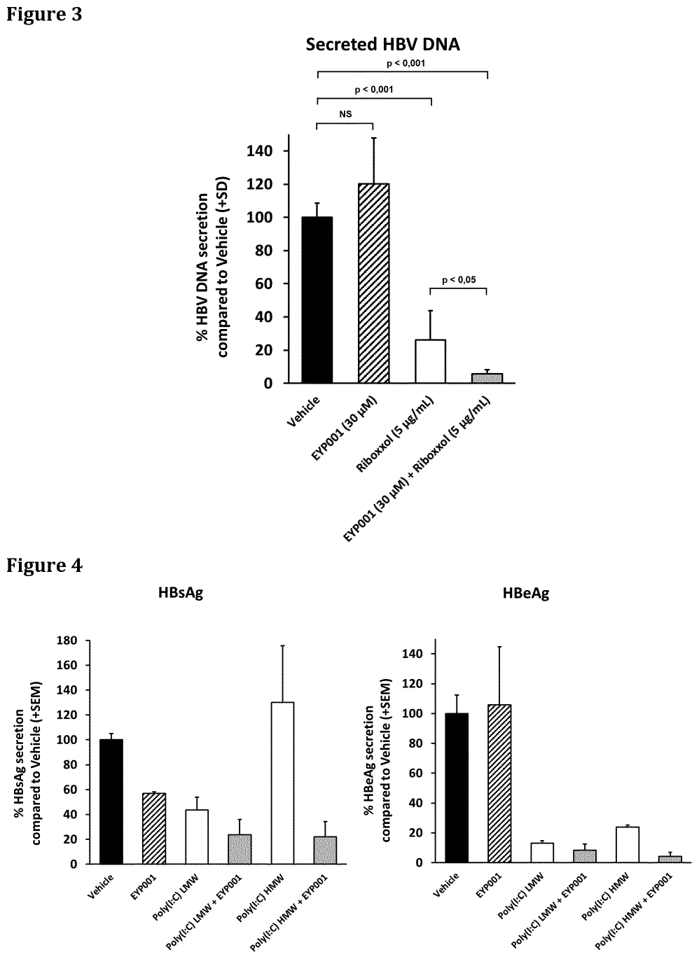

As shown on FIGS. 1, 2 and 3, the combination of an FXR agonist with a TLR3 agonist significantly and strongly improves the beneficial effects of the TLR3 agonists on HBsAg, HBeAg, and secreted HBV DNA levels. A synergistic effect of the combination of an FXR agonist with a TLR3 agonist can be observed. Those results demonstrate the potentiation of TLR3 agonism when combined with an FXR agonist.

Using the same protocol, HBV infected PHH were treated with the FXR agonist EYP001 at 10 μM, or with two other TLR3 agonists (Poly(I:C) Low Molecular Weight (LMW) at 25 μg/mL or Poly(I:C) High Molecular Weight (HMW) at 25 μg/mL), or with the combination of the FXR agonist EYP001 at 10 μM with each of the two TLR3 agonists at 25 μg/mL, or with the vehicle. As shown on FIG. 4, both combinations of the FXR agonist EYP001 with each of the two other TLR3 agonists strongly improve the beneficial effects of the two TLR3 agonists on HBsAg and HBeAg levels. Those data show that the synergy observed when combining an FXR agonist with the TLR3 agonist Riboxxol is also observed with two other TLR3 agonists.

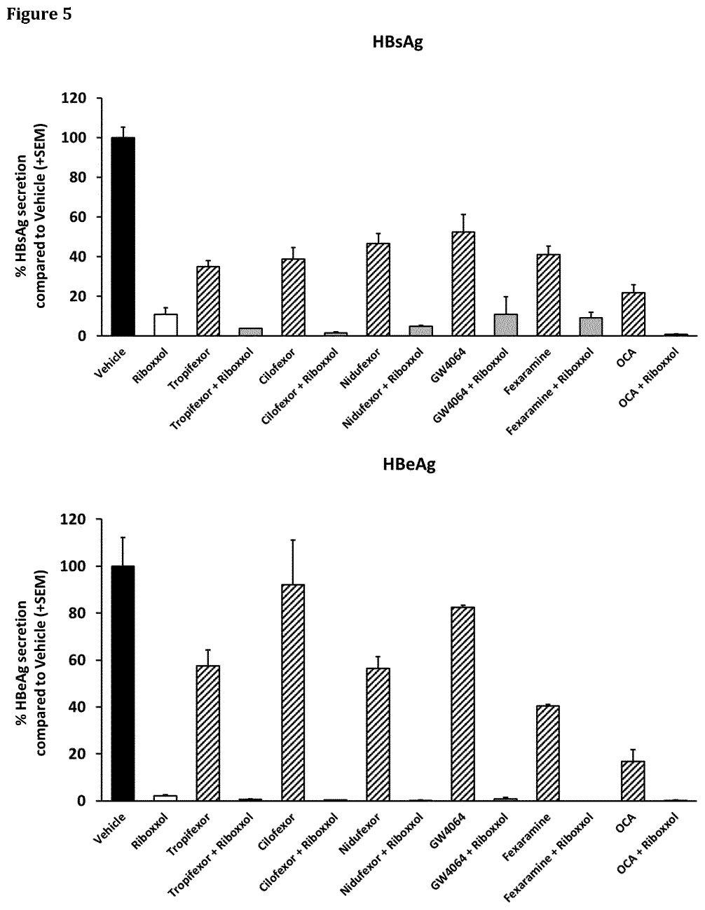

A third experiment was conducted the same way, in the same model of HBV infected PHH. In this experiment, 6 different FXR agonists (Tropifexor at 1 μM, Cilofexor at 10 μM, Nidufexor at 10 μM, GW4064 at 10 μM, Fexaramine at 10 μM and OCA at 10 μM) were combined with the TLR3 agonist Riboxxol at 5 μg/mL. As shown on FIG. 5, for both the HBsAg and HBeAg levels, all the FXR agonists tested improve the antiviral effects of the TLR3 agonist when combined.

In conclusion, a synergistic anti-HBV effect of the combination between an FXR agonist and a TLR3 agonist was observed for all the FXR agonists, and for all the TLR3 agonists evaluated.

1-21. (canceled)

22. A method of treating a hepatitis B virus (HBV) infection comprising administering a pharmaceutical composition comprising a farnesoid X receptor (FXR) agonist and a Toll-like receptor 3 (TLR3) agonist to a subject having an HBV infection; or
administering to a subject having an HBV infection a pharmaceutical composition comprising a farnesoid X receptor (FXR) agonist and a pharmaceutical composition comprising a Toll-like receptor 3 (TLR3) agonist; or
administering a pharmaceutical composition comprising a farnesoid X receptor (FXR) agonist to a subject having an HBV infection and being treated with a pharmaceutical composition comprising a Toll-like receptor 3 (TLR3) agonist; or
administering a pharmaceutical composition comprising a Toll-like receptor 3 (TLR3) agonist to a subject having an HBV infection and being treated with a pharmaceutical composition comprising a farnesoid X receptor (FXR) agonist.

23. The method according to claim 22, wherein the FXR agonist is selected from the group consisting of EYP001 (Vonafexor), LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502.

24. The method according to claim 22, wherein the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva).

25. The method according to claim 22, wherein the TLR3 agonist is a double stranded RNA compound (dsRNA).

26. The method according to claim 22, wherein the TLR3 agonist is selected from the group consisting of Poly I:C (polyribosinic:polyribocytidic acid), polyA:U (poly(adenylic acid-uridylic acid), Poly ICLC (polyinosinic acid-polycytidylic acid-poly-L-lysinecarboxy-methylcellulose complex or Hiltonol), PolyL:polyC₁₂U (polyIC₁₂U, Ampligen or Rintatolimod), Riboxxol (RGIC®50), RIBOXXIM (RGIC®100), APOXXIM, TL-532, ARNAX, IPH3102, MCT-465 and MCT-485.

27. The method according to claim 26, wherein the TLR3 agonist is a Poly I:C (polyribosinic:polyribocytidic acid).

28. The method according to claim 22, wherein the TLR3 agonist is selected from the group consisting of Riboxxol, Rintatolimod, and Hiltonol and the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva).

29. The method according to claim 22, wherein the TLR3 agonist is Riboxxol and the FXR agonist is selected from the group consisting of EYP001 (Vonafexor), LJN452 (Tropifexor), LMB763 (Nidufexor), GS-9674 (Cilofexor), GW4064, Fexaramine and OCA (Ocaliva).

30. The method according to claim 22, wherein the FXR agonist is EYP001 (Vonafexor) and the TLR3 agonist is Rintatolimod, Hiltonol or Riboxxol.

31. The method according to claim 22, wherein the FXR agonist is EYP001 (Vonafexor) and the TLR3 agonist is Riboxxol.

32. The method according to claim 22, wherein the HBV infection is a chronic HBV infection.
